(19) United States
(12) Patent Application Publication
(10) Pub. No.: US 2024/0029882 A1
(43) Pub. Date: Jan. 25, 2024

(54) DIAGNOSTIC CLASSIFICATION DEVICE AND METHOD

(71) Applicants: THE CATHOLIC UNIVERSITY OF KOREA INDUSTRY-ACADEMIC COOPERATION FOUNDATION, Seoul (KR); DELVINE INC., Seoul (KR)

(72) Inventors: Jae Woong LEE, Incheon (KR); Myungshin KIM, Seoul (KR); Yong Gu KIM, Seoul (KR); Sung Min CHO, Seoul (KR)

(21) Appl. No.: 18/039,566

(22) PCT Filed: Dec. 21, 2021
(86) PCT No.: PCT/KR2021/019494

§ 371 Date: May 31, 2023

(30) Foreign Application Priority Data
Dec. 24, 2020 (KR) 10-2020-0183149

(51) Int. Cl. G16H 50/20; G16B 25/10; G16B 40/20

(52) U.S. Cl. CPC G16H 50/20; G16B 25/10; G16B 40/20

(57) ABSTRACT

The present disclosure relates to a diagnostic classification device and method and, in particular, can provide a diagnostic classification device and method, which can provide an accurate diagnosis with only existing gene expression level measurement technology by extracting an expressed gene specifically expressed from gene expression level information about a patient and classifying a diagnosis name by using the expression level of the extracted expressed gene and artificial intelligence.

### TECHNICAL FIELD

The present embodiments provide a diagnostic classification device and method.

### BACKGROUND ART

Recently, with the development of information digitalization and data storage technology, a large amount of data has been accumulated, and artificial intelligence technology has been introduced and utilized in various fields. In particular, machine learning, a type of artificial intelligence technology, analyzes input data and probabilistically classifies objects or predicts values within a specific range, and is gradually being used in the medical field. Today, in the process of diagnosing complex diseases, such as leukemia, microscopic examination, chromosome examination, antigen examination, and fusion gene examination are comprehensively required, and new classification techniques, such as next generation sequencing (NGS), are being used. However, since the differential diagnosis process requires a variety of methods comprehensively, demand for time, effort, equipment, and cost continues to increase.

Further, in some cases, such as leukemia, there are a significant number of ambiguous cases that are not clearly classified in the classification system through routine methods, requiring various testing techniques to refine the diagnosis. Therefore, there is a need for differential diagnosis technology using artificial intelligence to provide accurate diagnosis using only existing gene expression measurement technology.

### DETAILED DESCRIPTION OF THE INVENTION

### Technical Problem

In the foregoing background, the present embodiments may provide a diagnostic classification device and method capable of classifying a diagnosis name from gene expression level information using artificial intelligence.

### Technical Solution

To achieve the foregoing objectives, in an aspect, the present embodiments provide a diagnostic classification device comprising a learning data generation unit extracting each expressed gene specifically expressed in a diagnosis name using gene expression amount information obtained from each patient group corresponding to a diagnosis name for each case and generating the expressed gene and an expression amount of the expressed gene according to the diagnosis name as learning data, a model training unit training a classification model for classifying the diagnosis name using the learning data, and a classification unit performing classification with the diagnosis name by applying new gene expression amount information to the classification model.

In another aspect, the present embodiments provide a diagnostic classification method comprising a learning data generation step extracting each expressed gene specifically expressed in a diagnosis name using gene expression amount information obtained from each patient group corresponding to a diagnosis name for each case and generating the expressed gene and an expression amount of the expressed gene according to the diagnosis name as learning data, a model training step training a classification model for classifying the diagnosis name using the learning data, and a classification step performing classification with the diagnosis name by applying new gene expression amount information to the classification model.

### BRIEF DESCRIPTION OF DRAWINGS

FIG. 1 is a view exemplarily illustrating a configuration of a system to which the disclosure may apply.

FIG. 2 is a view illustrating a configuration of a diagnostic classification device according to an embodiment of the disclosure.

FIG. 3 is a view illustrating an example for describing the operation of generating learning data in a diagnostic classification device according to an embodiment of the disclosure.

FIG. 4 is a view illustrating an example for describing the operation of classifying diagnosis names using a classification model in a diagnostic classification device according to an embodiment of the disclosure.

FIG. 5 is a view illustrating an example for describing a classification model in a diagnostic classification device according to an embodiment of the disclosure.

FIG. 6 is a view illustrating an example for describing the operation of verifying a classification model in a diagnostic classification device according to an embodiment of the disclosure.

FIG. 7 is a view illustrating an example for describing the operation of verifying a classification model in a diagnostic classification device according to another embodiment of the disclosure.

FIG. 8 is a flowchart illustrating a diagnostic classification method according to an embodiment of the disclosure.

### MODE FOR CARRYING OUT THE INVENTION

The disclosure relates to a diagnostic classification device and method.

Hereinafter, embodiments of the disclosure are described in detail with reference to the accompanying drawings. In assigning reference numerals to components of each drawing, the same components may be assigned the same numerals even when they are shown on different drawings. When determined to make the subject matter of the disclosure unclear, the detailed of the known art or functions may be skipped. The terms “comprises” and/or “comprising,” “has” and/or “having,” or “includes” and/or “including” when used in this specification specify the presence of stated features, regions, integers, steps, operations, elements, and/or components, but do not preclude the presence or addition of one or more other features, regions, integers, steps, operations, elements, components, and/or groups thereof. As used herein, the singular forms “a”, “an” and “the” are intended to include the plural forms as well, unless the context clearly indicates otherwise.

Such denotations as “first,” “second,” “A,” “B,” “(a),” and “(b),” may be used in describing the components of the present invention. These denotations are provided merely to distinguish a component from another, and the essence, order, or number of the components are not limited by the denotations.

In describing the positional relationship between components, when two or more components are described as “connected”, “coupled” or “linked”, the two or more components may be directly “connected”, “coupled” or “linked””, or another component may intervene. Here, the other component may be included in one or more of the two or more components that are “connected”, “coupled” or “linked” to each other.

When such terms as, e.g., “after”, “next to”, “after”, and “before”, are used to describe the temporal flow relationship related to components, operation methods, and fabricating methods, it may include a non-continuous relationship unless the term “immediately” or “directly” is used.

When a component is designated with a value or its corresponding information (e.g., level), the value or the corresponding information may be interpreted as including a tolerance that may arise due to various factors (e.g., process factors, internal or external impacts, or noise).

In the disclosure, fold change (FC) may mean a ratio of an original measurement and a subsequent measurement for describing how much the quantity is changed between the two measurements. Specifically, fold change (FC) may mean a value that is used to compare gene expression levels under two conditions and obtained by dividing a comparative condition (treatment) value by a reference condition (control) value.

Hereinafter, embodiments of the disclosure are described in detail with reference to the accompanying drawings.

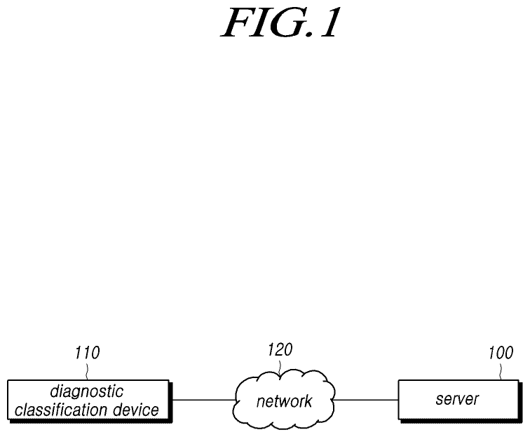

FIG. 1 is a view exemplarily illustrating a configuration of a system to which the disclosure may apply. Referring to FIG. 1, the disclosure relates to a system that provides a diagnostic classification method, and may be implemented in a diagnostic classification device 110 and a server 100.

The diagnostic classification device 110 may include a general PC, such as a general desktop or laptop computer, and may include a mobile terminal, such as a smartphone, a tablet PC, a personal digital assistant (PDA), and a mobile communication terminal, but without limitations thereto, should be broadly interpreted as any electronic device capable of communicating with the server 100.

The server 100 hardware-wise has the same configuration as a conventional web server or web application server or WAP server. However, the test control device 100 may softwarewise be implemented through any language, such as C, C++, Java, PHP, .Net, Python, Ruby, and may include program modules that perform various functions.

Further, the server 100 may be connected with a plurality of unspecified clients (including the device 110) and/or other servers through a network. Thus, the server 100 may be a computer system that receives a task performing request from a client or another server and derives and provides a result of the task, or computer software (server program) installed for such a computer system.

The server 100 should be understood as a concept that encompasses a series of application programs operated on the server 100 in addition to the above-described server programs and, in some case, various databases established inside or outside. Here, the database may mean a collection of data in which data, such as information, is structured and managed for the purpose of being used by the server or other devices, or may mean a storage medium storing such data collection. Further, such a database may include a plurality of databases classified according to a data structuring scheme, management scheme, type, and the like. In some cases, the database may include a database management system (DBMS), which is software that allows information or data to be added, corrected, or deleted.

Further, the server 100 may store and manage various types of information and data in a database. Here, the database may be implemented inside or outside the server 100.

Further, the server 100 may be implemented by way of a server program that is provided in various ways according to operating systems, such as DOS, Windows, Linux, UNIX, and Macintosh on general server hardware and, as a representative examples, may use a website used in the Windows environment or Internet information server (IIS), and Apache, Nginx, or Light HTTP used in the Unix environment.

Meanwhile, the network 120 is a network that connects the server 100 and the diagnostic classification device 110 and may be a closed network 120, such as local area network (LAN) or wide area network (WAN), or an open network, such as the Internet. The Internet may mean a global open computer network structure that provides the TCP/IP protocol and several services present on the higher layer, i.e., hypertext transfer protocol (HTTP), Telnet, file transfer protocol (FTP), domain name system (DNS), simple mail transfer protocol (SMTP), simple network management protocol (SNMP), network file service (NF S), or network information service (NIS).

The diagnostic classification device and method briefly described above are described below in more detail.

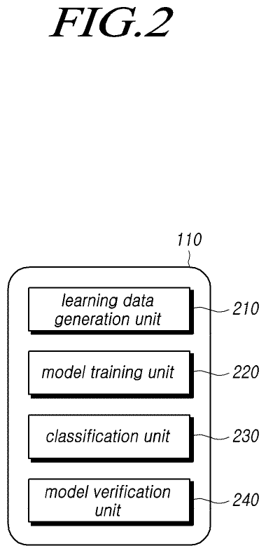

FIG. 2 is a view illustrating a configuration of a diagnostic classification device according to an embodiment of the disclosure. Referring to FIG. 2, a diagnostic classification device 110 according to an embodiment of the disclosure comprises a learning data generation unit 210 extracting each expressed gene specifically expressed in a diagnosis name using gene expression amount information obtained from each patient group corresponding to a diagnosis name for each case and generating the expressed gene and an expression amount of the expressed gene according to the diagnosis name as learning data, a model training unit 220 training a classification model for classifying the diagnosis name using the learning data, and a classification unit 230 performing classification with the diagnosis name by applying new gene expression amount information to the classification model.

The learning data generation unit 210 may extract each expressed gene specifically expressed for each diagnosis name using gene expression amount information obtained from each patient group corresponding to the diagnosis name for each case. For example, the learning data generation unit 210 may obtain gene expression amount information by analyzing mRNA of bone marrow cells or peripheral blood leukocytes reflecting the genotype of leukemia cells. The learning data generation unit 210 may use gene expression amount information measured from each patient group corresponding to acute myeloid leukemia (AML), acute lymphoblastic leukemia (ALL), and a mixed phenotype acute leukemia (MPAL). For example, gene expression amount information may be measured and obtained using an RNA sequencing (RNA-seq) method and a microarray method. However, this is an example, and is not limited thereto as long as it is a test method capable of measuring the amount of gene expression.

As another example, the learning data generation unit 210 may generate learning data by extracting the expressed gene from gene expression amount information corresponding to each diagnosis name. For example, the learning data generation unit 210 may first normalize gene expression amount information corresponding to the diagnosis name using a housekeeping gene, and extract the expressed gene by comparing the first normalized expression amount. Specifically, the learning data generation unit 210 may divide the expression amount of all the genes of the patient corresponding to the diagnosis name by the housekeeping gene to perform first normalization and compare the first normalized expression amount to extract the expressed gene that is specifically expressed. In this case, the housekeeping gene is tyrosine-protein kinase (ABL1) and may be a representative gene that is constantly expressed in all tissues regardless of conditions and seldom changes in expression amount. Accordingly, the learning data generation unit 210 may extract the expressed gene that is specifically expressed regardless of the condition by performing first normalization using the detection value of the housekeeping gene detected at the same time when detecting the mRNA.

As another example, the learning data generation unit 210 may extract a gene in which a difference in the median value of the first normalized expression amount is equal to or more than N fold change (FC), as the expressed gene. However, the learning data generation unit 210 may exclude a gene whose first normalized expression amount is less than or equal to a specific value from the extracted expressed gene. Specifically, the learning data generation unit 210 may extract a gene that exhibits a relatively high expression amount of 2 fold change (FC) or more based on the median value of the first normalized expression amount, as the expressed gene. Further, even if there is the statistical difference, the learning data generation unit 210 may technically exclude the gene having the first normalized expression amount less than or equal to the specific value, which has low reproducibility of the measured value, from the expressed gene. In this case, the specific value may be arbitrarily set based on the median value of the expression amount of all genes.

Further, the learning data generation unit 210 may generate the expression amount of the expressed gene extracted according to the diagnosis name for each case, as learning data. For example, the learning data generation unit 210 may perform second normalization on the expression amount of the expressed gene using the expression average value of all genes included in the gene expression amount information, and generate the second normalized expression amount as learning data. Specifically, the learning data generation unit 210 may generate learning data by performing the second normalization in a manner of dividing the expression amount of the expressed gene specifically expressed according to the diagnosis name by the expression average value of all the genes.

The model training unit 220 may train a classification model for classifying the diagnosis name using the generated learning data. For example, the model training unit 220 may calculate a difference between diagnosis names using a support vector machine (SVM) and generate a classification model that performs classification from gene expression amount information with the diagnosis name based on the difference. For example, the classification model may be a machine learning model that plots the learning data as a spot in a specific dimensional space and classifies the plotted dot based on a hyperplane. Specifically, the classification model may be a soft margin SVM model using a kernel function because the gene expression amount is not linearly separated according to the diagnosis name classification. Details of the classification model are described below with reference to FIG. 5.

The classification unit 230 may perform classification on the diagnosis name by applying new gene expression amount information to the classification model. For example, if the gene expression amount information of the new case is input, the classification unit 230 may perform classification on the diagnosis name by applying the trained machine learning model. This may provide an effect of classifying the diagnosis name by applying it to the classification model even when an ambiguous case in which it is not clearly classified by the classification system occurs.

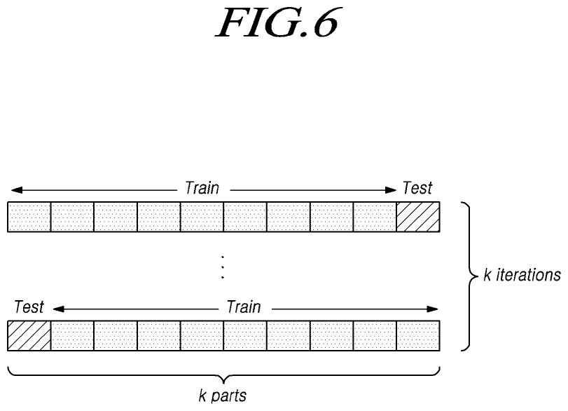

The model verification unit 240 may perform cross-verification to measure the performance of the classification model. For example, the model verification unit 240 may divide the learning data into K groups, redivide each group into K groups, and designate a learning set and a verification set to perform a verification process. In this case, in each group, the verification process may be repeatedly performed with the learning set and the verification set designated to differ from each other. Details of cross-verification are described below with reference to FIG. 6.

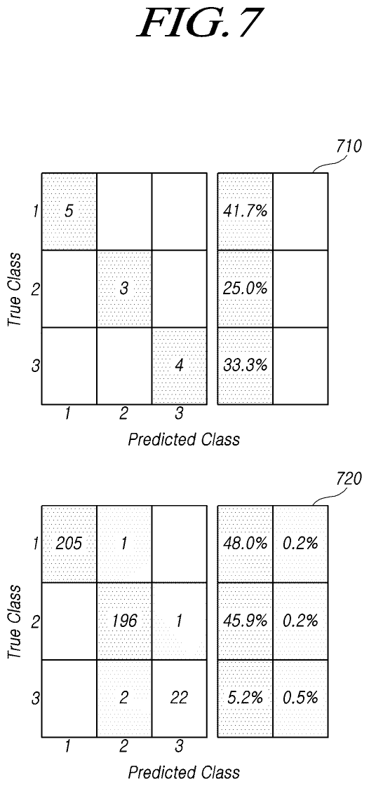

Further, the model verification unit 240 may generate a confusion matrix to measure the performance of the classification model. For example, the model verification unit 240 may generate a confusion matrix by comparing the verification result of the verification set with the actual diagnosis result, and determine the reliability of the classification model by calculating a prediction value based on the probability value of the confusion matrix. Details of the confusion matrix are described below with reference to FIG. 7.

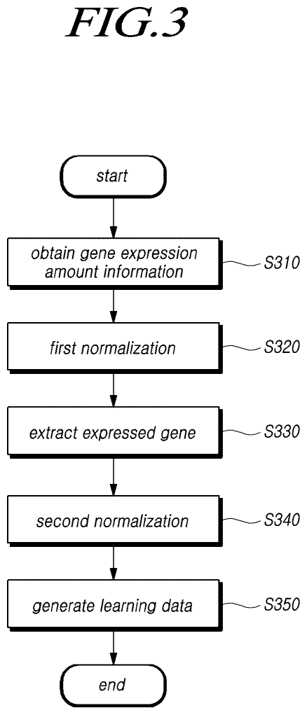

FIG. 3 is a view illustrating an example for describing the operation of generating learning data in a diagnostic classification device according to an embodiment of the disclosure. Referring to FIG. 3, the learning data generation unit 210 of the diagnostic classification device according to an embodiment of the disclosure may obtain gene expression amount information (S310). As an example, the learning data generation unit 210 may obtain gene expression amount information measured from each patient group corresponding to acute myeloid leukemia (AML), acute lymphoblastic leukemia (ALL), and a mixed phenotype acute leukemia (MPAL). For example, the learning data generation unit 210 may obtain gene expression amount information by measuring about 30,000 mRNAs in cells separated from the blood of each of the patient diagnosed with AML, the patient diagnosed with ALL, and the patient diagnosed with MPAL.

Further, the learning data generation unit 210 may use a microarray method or an RNA-seq method to measure gene expression amount information. For example, the microarray method may simultaneously measure the expression amount of thousands of genes and may statistically discover patterns that are expressed differently depending on the type of diagnosis. Further, the RNA-seq technique measures mRNA in cells using high-throughput sequencing, and may identify the degree of expression of each gene according to the type of diagnosis with the number of mapped reads. However, this is an example, and is not limited thereto as long as it is a method capable of measuring the amount of gene expression.

The learning data generation unit 210 may first normalize the gene expression amount information obtained according to each diagnosis name (S320). For example, the learning data generation unit 210 may first normalize the gene expression amount information corresponding to the diagnosis name using a housekeeping gene. For example, the learning data generation unit 210 may perform normalization by dividing the gene expression amount under each condition by the expression amount of the housekeeping gene and then compare expression amounts in order to compare the relative expression degrees of the genes under different conditions. In this case, the housekeeping gene is a gene expressed in all tissues or cells, unlike the expressed gene specifically expressed in the diagnosis name, and may be selected as a gene in which the expression difference between the expression tissues or cells is not more than twice. As a specific example, the housekeeping gene may be, but is not limited to, tyrosine-protein kinase (ABL1), glyceraldehyde-3-phosphate dehydrogenase (GAPDH), or the like.

The learning data generation unit 210 may extract the expressed gene specifically expressed according to the diagnosis name using the first normalized expression amount (S330). For example, the learning data generation unit 210 may extract a gene having a difference of 2 fold change (FC) or more based on the median value of the first normalized expression amount, as the expressed gene. For example, the expressed gene may be extracted using a value obtained by dividing the first normalized expression amounts by the median value. In this case, genes having an expression amount higher than the overall average expression amount may be aligned with values resultant from the division higher than 1. As another example, the learning data generation unit 210 may exclude genes in which the first normalized expression amount is less than or equal to a specific value based on the median value from the extracted expressed genes. For example, genes in which the value obtained by dividing the first normalized expression amounts by the median value is less than or equal to the specific value may be excluded from the extracted expressed genes. This is to exclude genes with very low expression from the expressed gene because the reproducibility of the measured value is technically low even if there is a statistical difference.

The learning data generation unit 210 may second normalize the expression amount of the extracted expressed gene using the expression average value of all genes included in the gene expression amount information (S340). For example, the learning data generation unit 210 may perform the second normalization in a manner of dividing the expression amount of the expressed gene specifically expressed in each diagnosis by the expression average value of all genes included in the corresponding diagnosis. Accordingly, the learning data generation unit 210 may increase the learning performance of the classification model by normalizing and inputting the expression amount of the extracted expressed gene. However, the corresponding step may be omitted as necessary.

The learning data generation unit 210 may generate an expressed gene and an expression amount of the expressed gene according to the diagnosis name as learning data (S350). For example, the learning data generation unit 210 may generate learning data by matching the diagnosis name for each case with the expressed gene specifically expressed in each diagnosis name and the expression amount of the corresponding expressed gene.

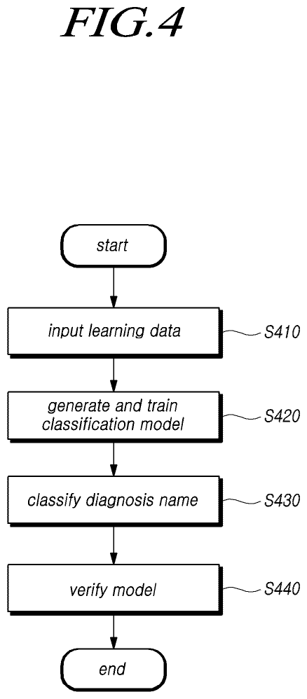

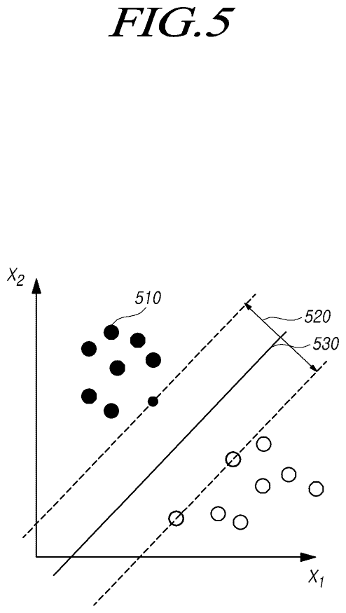

FIG. 4 is a view illustrating an example for describing the operation of classifying diagnosis names using a classification model in a diagnostic classification device according to an embodiment of the disclosure. Referring to FIG. 4, the learning data generation unit 210 of the diagnostic classification device according to an embodiment of the disclosure may input the generated learning data to the classification model (S410). For example, the learning data may be a database (DB) constructed by matching the specifically expressed gene extracted according to the diagnosis name for each case and the expression amount of the corresponding expressed gene with the diagnosis name for each case.

The model training unit 220 may generate a classification model for classifying the diagnosis name from gene expression amount information, and train the classification model using the learning data (S420). For example, the model training unit 220 may generate the classification model for classifying the diagnosis name by calculating the difference between diagnosis names from gene expression amount information using a support vector machine (SVM). Here, the classification model may be a map machine learning model using a classification algorithm for binary classification as the support vector machine. For example, the model training unit 220 may plot the expression amount information of the expressed gene according to each diagnosis name as a dot in a specific dimensional space, and classify the diagnosis name by dividing classes based on the hyperplane. In this case, the specific dimension may be set to the number of selected expressed genes, and the hyperplane may be set to maximize the distance from the hyperplane to the closest dot of each class.

The classification unit 230 may perform classification on the diagnosis name by applying the new gene expression amount information to the classification model (S430). For example, when the gene expression amount information of the new case is input, the classification unit 230 may apply it to the classification model and classify into diagnosis names corresponding to AML, ALL, and MPAL.

The model verification unit 240 may verify the classification model using the cross-verification or confusion matrix (S440). For example, when the number of verification sets for evaluating the performance of the classification model is small, the model verification unit 240 may verify the classification model using cross-verification. Accordingly, when the number of gene expression information corresponding to the diagnosis name for each case is small, the model verification unit 240 may verify the classification model using cross-verification.

As another example, the model verification unit 240 may verify the classification model using the confusion matrix to evaluate the performance by calculating the prediction value of the classification model. The model verification unit 240 may generate a confusion matrix to compare the verification result of the verification set with the actual diagnosis result, and may verify the classification model by calculating a prediction value based on the probability value. Here, the prediction value may be accuracy, precision, and recall.

FIG. 5 is a view illustrating an example for describing a classification model in a diagnostic classification device according to an embodiment of the disclosure. Referring to FIG. 5, a classification model generated by the model training unit 220 of the diagnostic classification device according to an embodiment of the disclosure may be described. For example, the classification model of the model training unit 220 may plot the learning data generated from the gene expression information as a dot 510 in a specific dimensional space. However, when it is difficult to linearly separate gene expression information, a process of optimizing classification using feature extraction and kernel functions may be required in the process of generating learning data.

For example, when the learning data is linearly separable, the model training unit 220 may use two parallel hyperplanes for classifying classes and having a maximum distance. In this case, the distance 520 of the margin is 2/∥w∥, and maximizing the distance 520 of the margin may be a target of the classification model. To that end, Equation 1 may be used. Further, the margin may mean the difference between the diagnosis names, and the class may mean the diagnosis name class.

min ½∥w∥²,s.t.yᵢ(w·xᵢ+b)≥1,i=1, . . . n [Equation 1]

Here, w and b may be coefficients of hyperplane, and xᵢ may be the dot (observed data point) plotted for learning data. Accordingly, the model training unit 220 may classify classes which have the same label as the predicted data.

For another example, when the learning data is not linearly separable, the model training unit 220 may use a soft margin support vector machine (SVM) which adds the slack variables (ζ). The model training unit 220 may add a value proportional to the distance from the hyperplane of each class toward the opposite class area to the objective function of finding the hyperplane 530 that maximizes the distance 520 of the margin, and may find the hyperplane that minimizes the value and maximizes the margin. The objective function for finding the optimal hyperplane is shown in Equation 2.

$$min \frac{1}{2} {‖ w ‖}^{2} + C {\sum }_{i = 1}^{m} {ζ}_{1} \\ s . t . {y}_{i} ( w \cdot ϕ ( {x}_{i} ) + b ) \geq 1 - {ζ}_{i} , i = 1 , … . n , {ζ}_{i} \geq 0 \qquad [ Equation 2 ]$$

Accordingly, the model training unit 220 may use a hyperbolic tangent among the sigmoid kernels as a kernel function used in the support vector machine, convert the dot 510 having the feature data in the two-dimensional space, and classify based on the hyperplane 530 having the maximum margin. The hyperbolic tangent kernel function may be expressed as equation 3.

k(xᵢ,xⱼ)=ϕ(xᵢ)·ϕ(xⱼ)

k(xᵢ,xⱼ)=tanh(αxᵢ·xⱼ=b) [Equation 3]

Here, xᵢ and xⱼ may be coordinates of the learning data, a>0, and b<0. Further, Φ(xⱼ) may be the converted learning data coordinates.

However, it has been described that the classification model uses a support vector machine, but it is an example, and is not limited as long as it is a model that classifies newly input data after trained with learning data such as a logistic regression method, a K nearest neighbor (KNN), and a decision tree.

FIG. 6 is a view illustrating an example for describing the operation of verifying a classification model in a diagnostic classification device according to an embodiment of the disclosure. Referring to FIG. 6, the model verification unit 240 of the diagnostic classification device according to an embodiment of the disclosure may perform cross-verification of the classification model. For example, the model verification unit 240 may perform a verification process of dividing the learning data generated from the gene expression amount information into K groups, redividing each group into K groups, and using one as the verification set while using the remaining k−1 as the learning set. However, the model verification unit 240 may perform the verification process with the learning set and the verification set designated to differ from each other in each group. The model verification unit 240 may average the result values obtained by repeating the verification process on the K groups and use it as the verification result value.

For example, when the model verification unit 240 uses 10-fold verification, the learning data may be composed of 10 groups. Further, the model verification unit 240 may divide the limited learning data into 10 sets by 9:1, and use one of the sets as the verification set while using the remaining nine sets as the learning set. In this case, the model verification unit 240 may set the respective verification sets of the 10 groups not to overlap each other. Further, since the gene expression information constituting the verification set is different for each repeated verification process, each result value of the model verification unit 240 may be calculated as different. Therefore, the model verification unit 240 may average the result values obtained through the verification process repeated 10 times and use the averaged value as the verification result value of the classification model. However, the 10 fold verification is described as an example, and the cross verification method is not limited thereto.

In other words, the model verification unit 240 may provide an effect of performing training and verification a total of k times using the limited training data.

FIG. 7 is a view illustrating an example for describing the operation of verifying a classification model in a diagnostic classification device according to another embodiment of the disclosure. Referring to FIG. 7, the model verification unit 240 of the diagnostic classification device according to an embodiment of the disclosure may determine the reliability of the classification model by generating a confusion matrix. For example, the model verification unit 240 may generate a confusion matrix consisting of the verification result (predicted class) of the verification set and the actual diagnosis result (true class). In this case, the labels written on the rows and columns of the confusion matrix may mean respective diagnosis names. Specifically, label 1 of the confusion matrix may be set to AML, label 2 to ALL, and label 3 to MPAL.

For example, the model verification unit 240 may generate the confusion matrix 710 using a result value learned from the local data using the classification model. Further, the model verification unit 240 may generate the confusion matrix 720 using a result value obtained by applying global data to the classification model learned from the local data. Accordingly, the model verification unit 240 may compare the two confusion matrices and determine whether the classification model generated as the local data reflects all the characteristics that may appear in the global data, thereby determining the reliability of the classification model.

As another example, the model verification unit 240 may determine the reliability of the classification model by calculating the prediction value based on the generated probability value of the confusion matrix. In this case, the prediction value may be accuracy, and the accuracy may be a criterion for evaluating whether the classification model actually accurately classifies gene expression information corresponding to AML, ALL, and MPAL as AML, ALL, and MPAL, respectively. For example, the accuracy may be calculated in a manner of dividing the number of cases in which the diagnosis result classified by inputting the verification set into the classification model and the actual diagnosis result are the same by the total number of cases input.

Hereinafter, a diagnostic classification method that may be performed by the diagnostic classification device described with reference to FIGS. 1 to 7 is described.

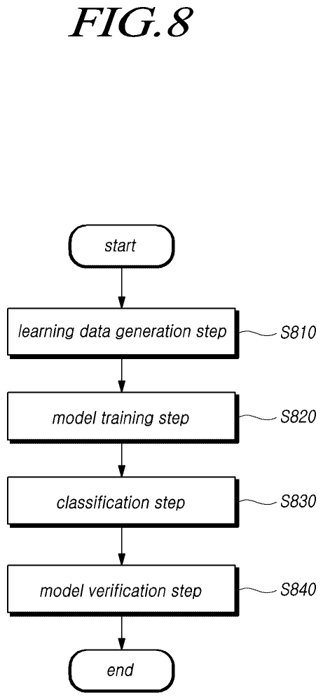

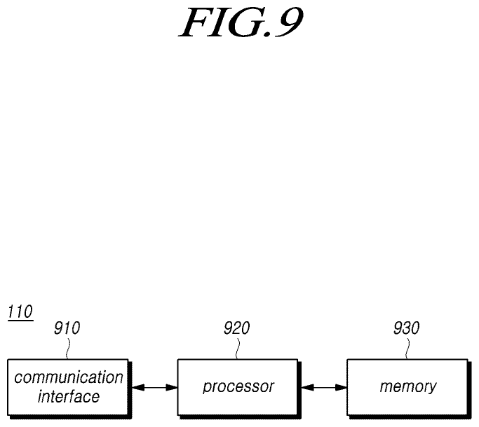

FIG. 8 is a flowchart illustrating a diagnostic classification method according to another embodiment of the disclosure. Referring to FIG. 8, the diagnostic classification method according to the disclosure may include a learning data generation step (S810). The diagnostic classification device may extract each expressed gene specifically expressed in the diagnosis name using gene expression amount information obtained from each patient group corresponding to the diagnosis name for each case. For example, the diagnostic classification device may obtain gene expression amount information by analyzing mRNA of bone marrow cells or peripheral blood leukocytes reflecting the genotype of leukemia cells. The diagnostic classification device may use gene expression amount information measured from each patient group corresponding to acute myeloid leukemia (AML), acute lymphoblastic leukemia (ALL), and a mixed phenotype acute leukemia (MPAL). For example, gene expression amount information may be measured and obtained using an RNA sequencing (RNA-seq) method and a microarray method. However, this is an example, and is not limited thereto as long as it is a test method capable of measuring the amount of gene expression.

As another example, the diagnostic classification device may generate learning data by extracting the expressed gene from gene expression amount information corresponding to each diagnosis name. For example, the diagnostic classification device may first normalize gene expression amount information corresponding to the diagnosis name using a housekeeping gene, and extract the expressed gene by comparing the first normalized expression amount. Specifically, the diagnostic classification device may divide the expression amount of all the genes of the patient corresponding to the diagnosis name by the housekeeping gene to perform first normalization and compare the first normalized expression amount to extract the expressed gene that is specifically expressed. In this case, the housekeeping gene is tyrosine-protein kinase (ABL1) and may be a representative gene that is constantly expressed in all tissues regardless of conditions and seldom changes in expression amount. However, ABL1 is an example of the housekeeping gene, and is not limited thereto if it corresponds to a housekeeping gene.

As another example, the diagnostic classification device may extract a gene in which a difference in the median value of the first normalized expression amount is equal to or more than N fold change (FC), as the expressed gene. However, the diagnostic classification device may exclude a gene whose first normalized expression amount is less than or equal to a specific value from the extracted expressed gene. Specifically, the diagnostic classification device may extract a gene that exhibits a relatively high expression amount of 2 fold change (FC) or more based on the median value of the first normalized expression amount, as the expressed gene. Further, even if there is the statistical difference, the diagnostic classification device may technically exclude the gene having the first normalized expression amount less than or equal to the specific value, which has low reproducibility of the measured value, from the expressed gene. In this case, the specific value may be arbitrarily set based on the median value of the expression amount of all genes.

Further, the diagnostic classification device may generate the expression amount of the expressed gene extracted according to the diagnosis name for each case, as learning data. For example, the diagnostic classification device may perform second normalization on the expression amount of the expressed gene using the expression average value of all genes included in the gene expression amount information, and generate the second normalized expression amount as learning data. Specifically, the diagnostic classification device may generate learning data by performing the second normalization in a manner of dividing the expression amount of the expressed gene specifically expressed according to the diagnosis name by the expression average value of all the genes.

The diagnostic classification method may include a model training step (S820). For example, the diagnostic classification device may train a classification model for classifying the diagnosis name using the generated learning data. For example, the diagnostic classification device may calculate a difference between diagnosis names using a support vector machine (SVM) and generate a classification model that performs classification from gene expression amount information with the diagnosis name based on the difference. Here, the classification model may be a machine learning model that plots the learning data as a spot in a specific dimensional space and classifies the plotted dot based on a hyperplane. Specifically, the classification model may be a soft margin SVM model using a kernel function because the gene expression amount is not linearly separated according to the diagnosis name classification.

The diagnostic classification method may include a classification step (S830). For example, the diagnostic classification device may perform classification on the diagnosis name by applying new gene expression amount information to the classification model. For example, if the gene expression amount information of the new case is input, the diagnostic classification device may perform classification on the diagnosis name by applying the trained machine learning model. This may provide an effect of classifying the diagnosis name by applying it to the classification model even when an ambiguous case in which it is not clearly classified by the classification system occurs.

The diagnostic classification method may include a model verification step (S840). For example, the diagnostic classification device may perform cross-verification to measure the performance of the classification model. For example, the diagnostic classification device may divide the learning data into K groups, redivide each group into K groups, and designate a learning set and a verification set to perform a verification process. In this case, in each group, the verification process may be repeatedly performed with the learning set and the verification set designated to differ from each other.

As another example, the diagnostic classification device may generate a confusion matrix to measure the performance of the classification model. For example, the diagnostic classification device may generate a confusion matrix by comparing the verification result of the verification set with the actual diagnosis result, and determine the reliability of the classification model by calculating a prediction value based on the probability value of the confusion matrix.

Although the diagnostic classification method according to an embodiment of the disclosure is described as being performed in a process as shown in FIG. 8, this is merely for convenience of description and, without departing from the essential concept of the disclosure, the steps may be performed in different orders, two or more steps may be integrated, or one step may be separated into two or more steps depending on implementations.

FIG. 9 is a block diagram illustrating a diagnostic classification device according to an embodiment. Referring to FIG. 9, a diagnostic classification device 110 according to an embodiment includes a communication interface 910 and a processor 920. The diagnostic classification device 110 may further include a memory 930. Each component, communication interface 910, processor 920, and memory 930 may be connected to each other through a communication bus. For example, the communication bus may include a circuit for connecting the components with one another and transferring communications (e.g., control messages and/or data) between the components.

The communication interface 910 may obtain gene expression amount information for each patient group corresponding to the diagnosis name for each case. Further, the communication interface 910 may perform communication with an external device through wireless communication or wired communication.

The processor 920 may perform at least one method described above in connection with FIGS. 1 to 8 or an algorithm corresponding to at least one method. The processor 920 may be a data processing device implemented in hardware having a circuit having a physical structure for executing desired operations. For example, the desired operations may include code or instructions included in a program. For example, the data processing unit implemented in hardware may include a microprocessor, a central processing unit, a processor core, a multi-core processor, a multiprocessor, a neural processing unit (NPU), an application-specific integrated circuit (ASIC), or a field programmable gate array (FPGA).

Further, the processor 920 may execute the program and may control the diagnostic classification device 110. The program code executed by the processor 920 may be stored in the memory 930.

Information about the artificial intelligence model including a neural network according to an embodiment of the disclosure may be stored in an internal memory of the processor 920 or may be stored in an external memory, that is, the memory 930. For example, the memory 930 may store gene expression amount information for each patient group corresponding to the diagnosis name for each case obtained through the communication interface 910. The memory 930 may store an artificial intelligence model including a neural network. Further, the memory 930 may store various information generated during processing by the processor 920 and output information extracted by the processor 920. The output information may be a neural network calculation result or a neural network test result. The memory 930 may store the neural network learning result. The neural network learning result may be obtained from the diagnostic classification device 110 or may be obtained from an external device. The neural network learning result may include weight and bias values. Further, the memory 930 may store various data and programs. The memory 930 may include a volatile memory or a non-volatile memory. The memory 930 may include a mass storage medium, such as a hard disk and the like, and may store various data.

The above-described embodiments are merely examples, and it will be appreciated by one of ordinary skill in the art various changes may be made thereto without departing from the scope of the present invention. Accordingly, the embodiments set forth herein are provided for illustrative purposes, but not to limit the scope of the present invention, and should be appreciated that the scope of the present invention is not limited by the embodiments. The scope of the disclosure should be construed by the following claims, and all technical spirits within equivalents thereof should be interpreted to belong to the scope of the disclosure.

### CROSS-REFERENCE TO RELATED APPLICATION

The instant patent application claims priority under 35 U.S.C. 119(a) to Korean Patent Application No. 10-2020-0183149, filed on Dec. 24, 2020, in the Korean Intellectual Property Office, the disclosure of which is herein incorporated by reference in its entirety. The present patent application claims priority to other applications to be filed in other countries, the disclosures of which are also incorporated by reference herein in their entireties.

1. A diagnostic classification device, comprising:
a learning data generation unit extracting each expressed gene specifically expressed in a diagnosis name using gene expression amount information obtained from each patient group corresponding to a diagnosis name for each case and generating the expressed gene and an expression amount of the expressed gene according to the diagnosis name as learning data;
a model training unit training a classification model for classifying the diagnosis name using the learning data; and
a classification unit performing classification with the diagnosis name by applying new gene expression amount information to the classification model.

2. The diagnostic classification device of claim 1, wherein the learning data generation unit obtains the gene expression amount information measured from each patient group corresponding to acute myeloid leukemia (AML), acute lymphoblastic leukemia (ALL), and mixed phenotype acute leukemia (MPAL).

3. The diagnostic classification device of claim 1, wherein the learning data generation unit performs first normalization on the gene expression amount information corresponding to the diagnosis name using a housekeeping gene and extracts the expressed gene by comparing the first normalized expression amount.

4. The diagnostic classification device of claim 3, wherein the learning data generation unit extracts a gene in which a difference in a median value of the first normalized expression amount is more than or equal to N fold change (FC) as the expressed gene, and wherein a gene in which the first normalized expression amount is less than or equal to a specific value is excluded from the expressed gene.

5. The diagnostic classification device of claim 1, wherein the learning data generation unit performs second normalization on the expression amount of the expressed gene using an expression average of all genes included in the gene expression amount information and generates the second normalized expression amount as the learning data.

6. The diagnostic classification device of claim 1, wherein the model training unit calculates a difference between diagnosis names using a support vector machine (SVM) and generates a classification model for performing classification with the diagnosis name from the gene expression amount information based on the difference, and wherein the classification model plots the learning data as a dot in a specific dimensional space and classifies the dot based on a hyperplane.

7. The diagnostic classification device of claim 1, further comprising a model verification unit dividing the learning data into K groups, re-dividing each group into K groups, and designating a learning set and a verification set to perform a verification process, wherein each group designates the learning set and the verification as different and repeatedly performs the verification process.

8. The diagnostic classification device of claim 7, wherein the model verification unit generates a confusion matrix by comparing a verification result of the verification set with an actual diagnosis result and calculates a prediction value based on a probability value of the confusion matrix to determine a reliability of the classification model.

9. A diagnostic classification method, comprising:
a learning data generation step extracting each expressed gene specifically expressed in a diagnosis name using gene expression amount information obtained from each patient group corresponding to a diagnosis name for each case and generating the expressed gene and an expression amount of the expressed gene according to the diagnosis name as learning data;
a model training step training a classification model for classifying the diagnosis name using the learning data; and
a classification step performing classification with the diagnosis name by applying new gene expression amount information to the classification model.

10. The diagnostic classification method of claim 9, wherein the learning data generation step obtains the gene expression amount information measured from each patient group corresponding to acute myeloid leukemia (AML), acute lymphoblastic leukemia (ALL), and mixed phenotype acute leukemia (MPAL).

11. The diagnostic classification method of claim 9, wherein the learning data generation step performs first normalization on the gene expression amount information corresponding to the diagnosis name using a housekeeping gene and extracts the expressed gene by comparing the first normalized expression amount.

12. The diagnostic classification method of claim 11, wherein the learning data generation step extracts a gene in which a difference in a median value of the first normalized expression amount is more than or equal to N fold change (FC) as the expressed gene, and wherein a gene in which the first normalized expression amount is less than or equal to a specific value is excluded from the expressed gene.

13. The diagnostic classification method of claim 9, wherein the learning data generation step performs second normalization on the expression amount of the expressed gene using an expression average of all genes included in the gene expression amount information and generates the second normalized expression amount as the learning data.

14. The diagnostic classification method of claim 9, wherein the model training step calculates a difference between diagnosis names using a support vector machine (SVM) and generates a classification model for performing classification with the diagnosis name from the gene expression amount information based on the difference, and wherein the classification model plots the learning data as a dot in a specific dimensional space and classifies the dot based on a hyperplane.

15. The diagnostic classification method of claim 9, further comprising a model verification step dividing the learning data into K groups, re-dividing each group into K groups, and designating a learning set and a verification set to perform a verification process, wherein each group designates the learning set and the verification as different and repeatedly performs the verification process.
